(19) United States
(12) Patent Application Publication
(10) Pub. No.: US 2012/0116241 A1
(43) Pub. Date: May 10, 2012

(54) PORTABLE ASTHMA DETECTION DEVICE AND STAND-ALONE PORTABLE ASTHMA DETECTION DEVICE

(75) Inventors: Dar-Bin Shieh, Tainan City (TW); Chyi-Her Lin, Tainan City (TW)

(73) Assignee: National Cheng Kung University, Tainan City (TW)

(21) Appl. No.: 13/290,238

(22) Filed: Nov. 7, 2011

Related U.S. Application Data
(60) Provisional application No. 61/410,504, filed on Nov. 5, 2010.

(51) Int. Cl. A61B 5/09; A61B 5/01; A61B 5/08

(52) U.S. Cl. 600/532; 600/539

(57) ABSTRACT

The present invention relates to a portable asthma detection device and a stand-alone portable asthma detection device. The portable asthma detection device includes a housing; a gas detection module comprising a turbine and a mini airflow sensor; a gas introducing tube; and a signal delivery unit. In addition, the stand-alone portable asthma detection device includes a housing; a gas detection module comprising a turbine and a mini airflow sensor; a gas introducing tube; at least one input unit; at least one display unit; and at least one delivery unit. Thus, the detection of breathing condition can be performed anytime and anywhere by using the portable asthma detection device and stand-alone device, and then the efficiency of monitor and management of asthma can be improved.

### CROSS REFERENCE TO RELATED APPLICATION

This application claims the benefit of filing date of U.S. Provisional Application Ser. No. 61/410,504, entitled “Asthma Scan Device” filed Nov. 5, 2010 under 35 USC §119(e)(1).

### BACKGROUND OF THE INVENTION

1. Field of the Invention

The present invention relates to a portable asthma detection device and, more particularly, to a portable asthma detection device being in cooperation with a smart phone, enabling the patient under test to obtain a real-time detection result of the asthma detection.

2. Description of Related Art

Asthma is a disease caused by the chronic inflammation of the tracheal mucosa, or the excess-sensitive of the tracheal. When the inflamed or sensitive tracheal is stimulated, the tracheal muscle will repeatedly contracted, and more mucus will be secreted, resulting in the airway obstruction, leading to the syndromes, such as coughing, chest pain, and the difficulty in breathing.

Since the asthma is a high risk and, not-curable disease, the breathing condition of the patient must be long-term monitored, in order to control and minimize the morbidity of an asthma attack. In general, the amount of the airflow or the chemical element in the gas obtained from the breathing of the patient can be obtained from a breathing system monitoring apparatus. Then, basing on these detection results, doctors can determine the body condition of the patient accurately, and then provide proper drug for curing the asthma disease. In this way, the morbidity of an asthma attack is minimized.

However, since the aforementioned breathing system monitoring apparatus is large-sized and extremely expensive, the patient must attend the hospital periodically for being detected by the breathing system monitoring apparatus, in order to keep updated on his/her breathing condition. The successively travel to and from the hospital not only costs lot of money, but also increases the morbidity of an asthma attack. Due to these unfavorable reasons, some patient cannot be frequently monitored for a long period of time. Currently, some kinds of home-based detecting apparatus have been developed. However, these home-based detecting apparatus still have problems like non-portable and lack of suitable detection functions. Therefore, even with these home-based detecting apparatus, the medical personnel still cannot obtain the long-term breathing monitoring result of the asthma patient, resulting in the unawareness to the current health condition of the patient.

Therefore, a portable asthma detection device, which is simple and capable of executing the asthma detection anytime and anywhere, is required. With the portable asthma detection device, the patient can detect his/her breathing at home or at any location. Besides, after being in cooperation with a smart phone, both the patient and the medical personnel can understand the patient's current breathing condition, facilitating them to monitor the patient's breath anytime and anywhere. In this way, the morbidity of the asthma attack is minimized.

### SUMMARY OF THE INVENTION

The main object of the present invention is to provide a portable asthma detection device, enabling the asthma patient to detect his/her breathing condition anytime and anywhere, and obtain the complete breathing diagram and the physical/chemical characteristics of the gas of his/her breathing.

Another object of the present invention is to provide a stand-alone portable asthma detection device, enabling the patient to detect his/her breathing condition anytime and anywhere, and obtain the simple breath response immediately.

The still another object of the present invention is to provide a portable asthma detection device, capable of integrating with the drug delivery module, in order to deliver the drug to the mouth of a patient at the time when an asthma attack of the patient is detected, thus relieving the asthma attack of the patient at the real-time.

The other object of the present invention is to provide a portable asthma detection device, in cooperation with a smart phone, which provides the positioning function and the wireless transmission function, for transmitting the detection result to the medical personnel or a remote monitoring system, enabling the medical personnel to execute a long-term monitoring and obtain a complete data information, in order to effectively control and minimizing the morbidity of the asthma attack.

To achieve the object, the portable asthma detection device of the present invention comprises: a housing; a gas detection module, which comprising: a turbine; and a mini airflow sensor, for detecting the rotation of the turbine; a gas introducing tube, connecting with the housing and introducing the gas into the gas detection module; and a signal delivery unit, connecting with the housing mutually.

In addition, a stand-alone portable asthma detection device is also provided in the present invention, which comprises: a housing; a gas detection module, which comprising: a turbine; and a mini airflow sensor, for detecting the rotation of the turbine; a gas introducing tube, connecting with the housing and introducing the gas into the gas detection module; at least one input unit, being installed on the housing; at least one display unit, being installed on the housing and used for displaying a detection result; and at least one delivery unit, for providing a positioning function and a wireless network function.

The portable asthma detection device of the present invention can further comprise a reduced-size type microprocessor, wherein the microprocessor comprises: a delivery connecting unit, for connecting the signal delivery unit with the microprocessor, enabling the delivery of the electronic signal output from the gas detection unit, to the microprocessor; a computing module, for transforming the electronic signal into a detection result; and at least one display unit, for displaying the detection result. Wherein, the microprocessor executing the data computing process and data transmission process is preferably a smart phone, such as an iPhone, an Android, and a Symbian, but not limited to the these smart phones. In addition, application software for detecting asthma can be installed onto or removed from the smart phone. The microprocessor can further include at least one system selected from the group consisting of a position system, a wireless transmission system, and a remote monitoring system.

Besides, the portable asthma detection device of the present invention can further include at least one selected from the group consisting of an electronic nose, a temperature sensor, and a gas detector. The electronic nose is connected with the gas introducing tube for detecting the chemical element and the gas molecule in the gas, wherein the gas molecule can be the nitric oxide, the carbon monoxide, or the carbon dioxide. The temperature sensor can be connected with the gas introducing tube for detecting the temperature of the gas. The gas detector may include a thermoelectric cooler or at least one liquid analyzing chip for cooling and condensing the gas with the thermoelectric cooler, then analyzing the chemical element in the gas with the liquid analyzing chip. Besides, the portable asthma detection device of the present invention can further include a drug delivery module, which includes a drug container and a motor. The motor can drive the turbine to rotate reversely for delivering the drug stored in the drug container to the gas introducing tube, then to the mouth of the patient. Furthermore, the stand-alone portable asthma detection device of the present invention can also selectively include the elements mentioned above, for providing much more flexibility in the asthma detection executed by the stand-alone portable asthma detection device of the present invention.

In the present invention, the gas detection module can be an electro-magnetic type sensing module, an optical coupling type sensing module, or a power-generating type sensing module. The gas detection module is preferably an electro-magnetic type sensing module, capable of preventing the chemical pollution and reducing the amount of the electricity required in the asthma detection. Thus, during the operation of the portable asthma detection device of the present invention, the power required is significantly saved and the safety of the operation is also raised.

In the portable asthma detection device of the present invention, the gas introducing tube, the gas detection module, and/or the signal delivery unit are integrated with the housing, for improving the portability of the portable asthma detection device of the present invention and facilitating the patient to bring the portable asthma detection device of the present invention along with him/her. In addition, the signal delivery unit can be designed as a foldable element, and a notch can be installed at the exterior side of the housing for receiving the signal delivery unit therein. Thus, the total size of the portable asthma detection device of the present invention can be minimized. Moreover, for enabling the patient to observe the detection result of his/her breathing dynamic on a display unit, when the detection is in progress, a wire can be installed between the signal delivery unit and the housing.

The power of the mobile phone can be provided to the portable asthma detection device of the present invention, through the signal delivery unit. Thus, the portable asthma detection device of the present invention can operate without the need of an exterior power source. In addition, since the elements of the portable asthma detection device of the present invention are integrated with each other, and no exterior power supply unit is required to exchange, the failure rate of the portable asthma detection device of the present invention is minimized. Besides, with the positioning function, the wireless transmission function, and the remote controlling function provided by the smart phone, and with the ability of installing and removing of the application software required in the asthma detection on-demand, the portable asthma detection device of the present invention can provide a complete detection result of the breathing of the patient, together with the position information and detecting time of the asthma detection, to the medical personnel. Thus, the medical personnel can obtain the breathing diagram, the maximum flow rate of the airflow, the average flow rate of the airflow, the peak flow rate of the airflow, or the slope of the breathing diagram, for improving the efficiencies of both the monitoring and the managing of the asthma disease.

Therefore, the portable asthma detection device and the stand-alone portable asthma detection device provided in the present invention enables the asthma patient to detect his/her breathing condition anytime and anywhere, and obtain the complete breathing diagram and the physical/chemical characteristics of the gas of his/her breathing. In addition, the portable asthma detection device and the stand-alone portable asthma detection device provided in the present invention can transmit the detection result to the medical personnel or a remote monitoring system, enabling the medical personnel to improving the efficiencies of both the monitoring and the managing of the asthma disease. In this way, the long-term and complete monitoring, along with the curing on the asthma attack of the patient can be achieved, thus further minimizing the morbidity of the asthma attack.

Other objects, advantages, and novel features of the invention will become more apparent from the following detailed description when taken in conjunction with the accompanying drawings.

### BRIEF DESCRIPTION OF THE DRAWINGS

FIG. 1A is a schematic diagram displaying the optical coupling type sensing module of the present invention.

FIG. 1B is a schematic diagram displaying the electro-magnetic type sensing module of the present invention.

FIG. 1C is a schematic diagram displaying the power-generating type sensing module of the present invention.

FIG. 2A is a schematic diagram displaying the background noise during the execution of the asthma detection.

FIG. 2B is a schematic diagram displaying the normal asthma detection result background noise during the execution of the asthma detection.

FIG. 2C is a schematic diagram displaying the accurate breathing diagram from the asthma detection.

FIG. 3A is a schematic diagram displaying the portable asthma detection device in the yet-assembled status, according to the first embodiment of the present invention.

FIG. 3B is a schematic diagram displaying the portable asthma detection device in the assembled status, according to the first embodiment of the present invention.

FIG. 4 is a schematic diagram displaying the portable asthma detection device, in cooperation with a smart phone, according to the first embodiment of the present invention.

FIG. 5A is a schematic diagram displaying the portable asthma detection device in the receiving status, according to the second embodiment of the present invention.

FIG. 5B is a schematic diagram displaying the portable asthma detection device in the yet-receiving assembled status, according to the second embodiment of the present invention.

FIG. 6 is a schematic diagram displaying the portable asthma detection device, in cooperation with a smart phone, according to the second embodiment of the present invention.

FIG. 7A is a schematic diagram displaying the portable asthma detection device in the receiving status, according to the third embodiment of the present invention.

FIG. 7B is a schematic diagram displaying the portable asthma detection device in the yet-receiving status, according to the third embodiment of the present invention.

FIG. 8 is a schematic diagram displaying the portable asthma detection device, in cooperation with a smart phone, according to the third embodiment of the present invention.

FIG. 9A is a schematic diagram displaying the portable asthma detection device in the receiving status, according to the fourth embodiment of the present invention.

FIG. 9B is a schematic diagram displaying the portable asthma detection device in the yet-receiving status, according to the fourth embodiment of the present invention.

FIG. 10 is a schematic diagram displaying the portable asthma detection device, in cooperation with a smart phone, according to the fourth embodiment of the present invention.

FIG. 11 is a schematic diagram displaying the interior of the gas detector.

FIG. 12A and FIG. 12B are the schematic diagrams displaying the stand-alone portable asthma detection device of the present invention.

### DETAILED DESCRIPTION OF THE PREFERRED EMBODIMENT

The present invention provides a portable asthma detection device, which can achieve tasks as computing, executing, and transmitting, in cooperation with the microprocessor of a smart phone. Thus, the portable asthma detection device provided in the present invention can execute the breathing detection anytime and anywhere.

The portable asthma detection device of the present invention comprises: a housing, a gas detection module, a gas introducing tube, and a signal delivery unit. Wherein, the gas detection module includes a turbine and a mini airflow sensor. When the patient under test blows air toward the gas introducing tube, the airflow is introduced to the gas detection module, through the gas introducing tube. At the mean time, the turbine rotates with the flowing of the airflow. The mini airflow sensor detects the rotation of the turbine and outputs an electronic signal corresponding to the rotation of the turbine. Then, the signal delivery unit, such as a TSSR connecting terminal, and the delivery connecting unit deliver the electronic signal output from the mini airflow sensor to the smart phone, for executing further processing process and computing process.

In the portable asthma detection device of the present invention, the gas detection module may have 3 different types, i.e. the electro-magnetic type sensing module, the optical coupling type sensing module, and the power-generating type sensing module. Wherein, the optical coupling type sensing module can be designed as a chemical-pollution-free sensing module. Besides, the power-generating type sensing module can be designed as a low-power-required sensing module. In addition, the electro-magnetic type sensing module can be designed as a sensing module both chemical-pollution-free and low-power-required.

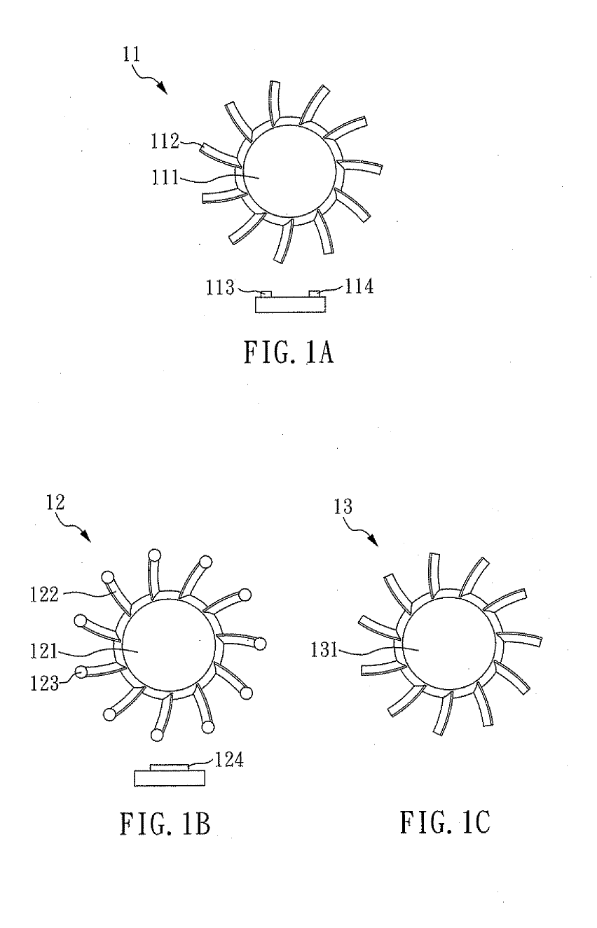

Please refer to FIG. 1A, which is a schematic diagram displaying the optical coupling type sensing module of the present invention. The optical coupling type sensing module 11 includes a turbine 111, and a detection unit having a light source 113 and a light sensor 114. When the light is emitted to the fan 112 of the turbine 111, the light sensor 114 can detect the light reflected by the fan 112. By detecting the variation of the amount of the reflected light with the light detector 114, both the flowing rate of the airflow and the amount of the airflow blew by the patient under test can be computed and thus be obtained. In this way, the breathing condition of the patient under test can be detected.

Please refer to FIG. 1B, which is a schematic diagram displaying the electro-magnetic type sensing module of the present invention. The electro-magnetic type sensing module 12 at least includes a magnet 123 installed on the fan 122 of the turbine 121, and an electro-magnetic sensor 124 installed on the housing near the fan 122 of the turbine 121. When the patient under test blows air toward the gas introducing tube, the airflow is introduced to the electro-magnetic type sensing module 12, through the gas introducing tube. At the mean time, by detecting the variation of the magnetic flux caused by the rotation of the fan 122 with the electro-magnetic sensor 124, the rotation velocity of the turbine 121, caused by the flowing of the airflow, is thus detected, Then, both the flowing rate of the airflow and the amount of the airflow blew by the patient under test can be computed and thus obtained. In addition, the installing position of the magnet and electro-magnetic sensor can be exchanged. That is, in some applications, the electro-magnetic sensor is installed on the fan, while the magnet is installed on the housing near the fan. Even with different arrangement of the magnet and the electro-magnetic sensor, same detection result will obtained as the detection result obtained previously.

Please refer to FIG. 1C, which is a schematic diagram displaying the power-generating type sensing module of the present invention. In the power-generating type sensing module 13, a power generator (not shown in the figure) is connected with the rotation axis of the turbine. When the patient under test blows air toward the gas introducing tube, the airflow is introduced to the power-generating type sensing module 13, through the gas introducing tube. At the mean time, the turbine 131 rotates with the flowing of the airflow, and the power generator (not shown in the figure) connected with the rotation axis of the turbine also rotates with the rotation of the turbine 131. By detecting the magnitude of the electricity generated by the power generator, and the instantaneous signal, both the flowing rate of the airflow and the amount of the airflow blew by the patient under test can be computed and thus be obtained. In this way, the breathing condition of the patient can be detected.

Besides, the portable asthma detection device of the present invention can selectively be in cooperation with a smart phone, for data processing purpose and thus obtaining a detection result. The smart phone can include a display unit, a delivery connecting unit, and a computing module. In addition, the smart phone can selectively include the functions of positioning, wireless transmission, and remote controlling. Moreover, the application software required by the asthma detection can be installed onto or removed from the smart phone on demand. When the smart phone receives the electronic signal, the electronic signal is transformed into a detection result with any kind of suitable computing module. Then, the detection result is displayed on the display unit in the form of diagram or data. Thus, the portable asthma detection device of the present invention can continuously detect and display the variation of the flowing rate of the airflow of the patient under test, during the whole asthma detection process. Therefore, the portable asthma detection device of the present invention can provide the complete detection result of the breathing condition, for obtaining the breathing diagram of the patient under test, the maximum flow rate of the airflow, the average flow rate of the airflow, the peak flow rate of the airflow, or the slope of the breathing diagram, which represents the current breathing condition of the patient under test.

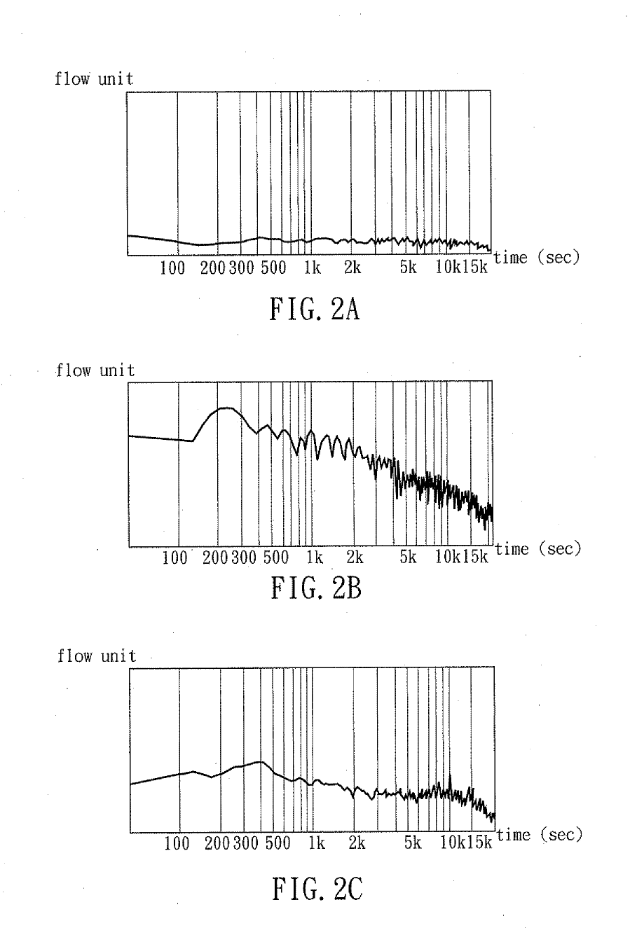

Moreover, through the wireless network function and the positioning function provided by the smart phone, the environmental factors of the geographic position of the asthma detection can be obtained, such as the temperature, the humidity, and the air pollution indices, etc. Thus, the computing module of the smart phone can compute the data, with the introduction of the background noise (as shown in FIG. 2A), with the introduction of the aforementioned environmental factors, with the introduction of the detection data obtained from a normal asthma detection (as shown in FIG. 2B), and/or with the introduction of the detection data obtained from a person doesn't have the asthma disease, in order to obtain an accurate breathing diagram from the asthma detection (as shown in FIG. 2C). Moreover, the smart phone can transmit the detection result to the specific hospital, the specific medical personnel, or the remote monitoring system, along with the geographical position and the time information of the asthma detection. Thus, the receiving end of the detection result can integrate the records in the database, with the environmental factors of the position where the asthma detection being executed. Then, by executing the long-term and frequent monitoring on the breathing condition of the patient, the efficiency of the monitoring and managing of the breathing condition of the patient can be increased.

In addition, through the signal delivery unit, the smart phone can provide electric power to the portable asthma detection device of the present invention. Thus, no external power source is required during the operation of the portable asthma detection device of the present invention. Which means, none of the problem related to the exchange of the power source will be raised. As a result, the portable asthma detection device of the present invention is high facilitated, multi-functional, low-power-related risk, and having low mechanical failure rate.

The embodiments provided below are merely used to provide some of the possible kinds of the portable asthma detection device of the present invention. In should be noticed that these embodiments are not used to limit the scope of the present invention.

### Embodiment 1

In the first embodiment, i.e., the embodiment 1, the signal delivery unit 35 and the gas detection module are integrated with the housing 31, wherein a notch 34 is installed on the housing 31. The signal delivery unit 35 is a foldable element, enabling the signal delivery unit 35 to be folded and then received in the notch 34, basing on the application situation. Thus, the total size of the portable asthma detection device of the present invention is minimized, enabling the patient to bring the portable asthma detection device along with him/her. In addition, the signal delivery unit 35 is also protected from any possible damage.

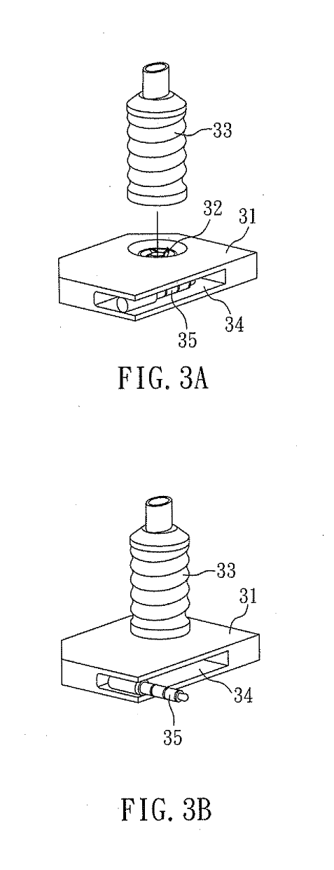

Please refer to FIG. 3A and FIG. 3B, wherein FIG. 3A is a schematic diagram displaying the portable asthma detection device in the yet-assembled status, according to the first embodiment of the present invention; while FIG. 3B is a schematic diagram displaying the portable asthma detection device in the assembled status, according to the first embodiment of the present invention. When the portable asthma detection device of the present invention is not yet used in the asthma detection, the housing 31 and the gas introducing tube 33 can be stored separately. Besides, for preventing the patient being affected with any possible kind of pollution, the gas introducing tube 33 is stored in a clean container. When the portable asthma detection device of the present invention is going to be used in the asthma detection, the gas introducing tube 33 is mutually engaged with the housing 31. In this way, when the patient under test blows air toward the inlet of the gas introducing tube 33, the airflow can be introduced to the gas detection module (not shown in the figure), through the gas introducing tube 33, enabling the turbine 32 to rotate with the flowing of the airflow.

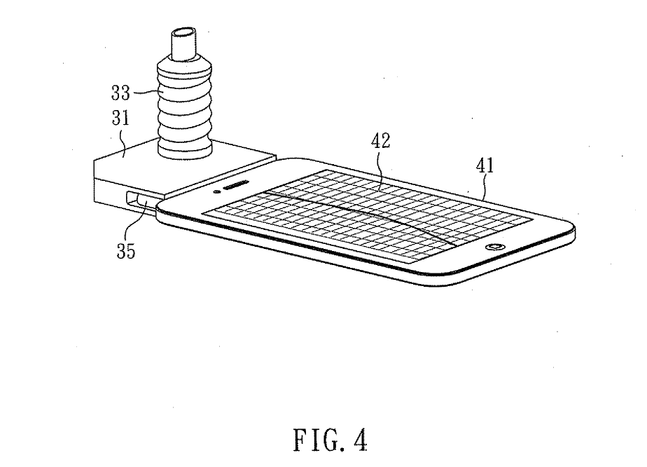

Please refer to FIG. 4, which is a schematic diagram displaying the portable asthma detection device, in cooperation with a smart phone, according to the first embodiment of the present invention. In the present embodiment, the signal delivery unit 35 of the portable asthma detection device of the present invention is connected with the delivery connecting unit of the smart phone (not shown in the figure). The smart phone 41 receives the electronic signal output from the mini airflow sensor. Then, after executing the process of computing with the hardware and software, the process of data comparison, and the process of outputting the detection result, the smart phone 41 displays the detection result, including the breathing diagram, the amount of the airflow, the breathing times, or the frequency of the breathing, on the display unit 42 thereof.

### Embodiment 2

In the second embodiment, i.e., the embodiment 2, another form of the portable asthma detection device of the present invention is provided. In this embodiment, the gas introducing tube 33, the gas detection module, and the signal delivery unit 35 are integrated with the housing 31. In addition, an electronic nose 51 is installed between the gas introducing tube 33 and the gas detection module, for detecting the chemical element, the kinds of the gas (for example, the nitric oxide, and the carbon dioxide, etc), and the concentration of these chemical element in the airflow.

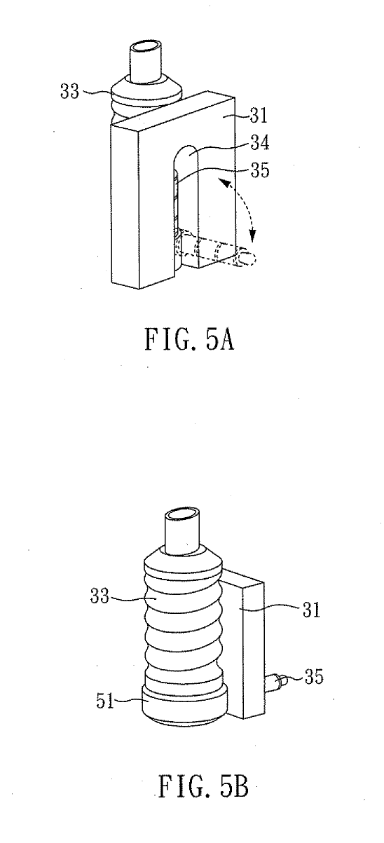

Please refer to FIG. 5A and FIG. 5B, wherein FIG. 5A is a schematic diagram displaying the portable asthma detection device in the receiving status, according to the second embodiment of the present invention; while FIG. 5B is a schematic diagram displaying the portable asthma detection device in the yet-receiving status, according to the second embodiment of the present invention. In the present embodiment, the electronic nose 51 is installed at the end of the gas introducing tube 33. Besides, a notch 34 can also be installed on the housing 31. The signal delivery unit 35 can be a foldable element. Thus, the signal delivery unit 35 can be folded and then received in the notch 34, when the portable asthma detection device of the present invention is not yet used in the asthma detection. Thus, the total size of the portable asthma detection device of the present invention is minimized, resulting in the increasing of the portability of the portable asthma detection device of the present invention.

When the portable asthma detection device of the present invention is going to be used in the asthma detection, the signal delivery unit 35 is extended outwardly, making the signal delivery unit 35 to be protruded from the notch 34 of the housing 31. Thus, the signal delivery unit 35 can be connected with the smart phone mutually for delivering the electronic signal to the smart phone, in order to execute further processing process and computing process.

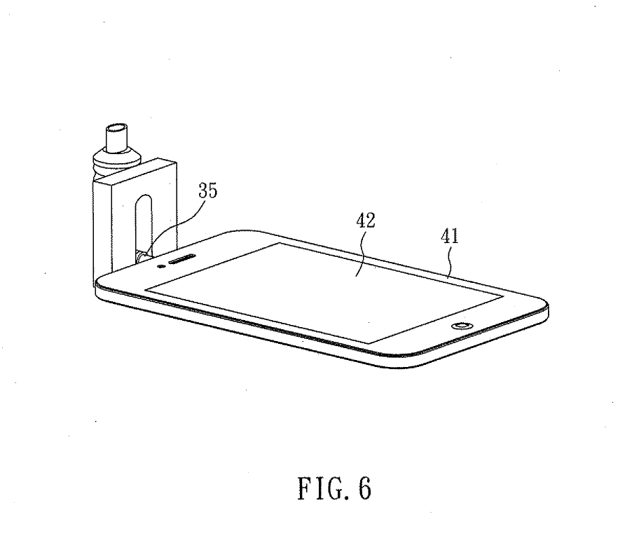

Please refer to FIG. 6, which is a schematic diagram displaying the portable asthma detection device, in cooperation with a smart phone, according to the second embodiment of the present invention. In the present embodiment, the signal delivery unit 35 of the portable asthma detection device of the present invention is connected with the delivery connecting unit of the smart phone (not shown in the figure). Thus, the smart phone 41 can execute the process of computing with the hardware and software, the process of data comparison, and the process of outputting the detection result, and then display the detection result, including the breathing diagram, the amount of the airflow, the breathing times, or the frequency of the breathing, on the screen thereof. Besides, in the present embodiment, the portable asthma detection device can display the detection result, such as the chemical element or the containing element of the gas detected by the electronic nose, on the display unit 42 thereof. Then, the detection result can be transmitted to the database, the hospital, or the medical personnel, through the wireless transmission function and the positioning function provided by the smart phone, for obtaining a complete, comprehensive, and efficient monitoring and managing.

### Embodiment 3

In the third embodiment, i.e., the embodiment 3, another form of the portable asthma detection device of the present invention is provided. In this embodiment, the portable asthma detection device of the present invention further comprises a wire installed between the signal delivery unit 35 and the housing 31, enabling the signal delivery unit 35 to be connected with the housing 31 through the wire having a certain length. With the installation of the wire, the patient under test can observe the detection result of his/her breathing dynamic on a display unit, when the detection is in progress. Besides, in the present embodiment, a protecting cover 51 can be installed at the inlet of the gas introducing tube 33. In addition, a wire protecting cover 52 can also be installed at the end of the housing 31, for receiving the wire connecting the interior of the housing 31 and the signal delivery unit 35.

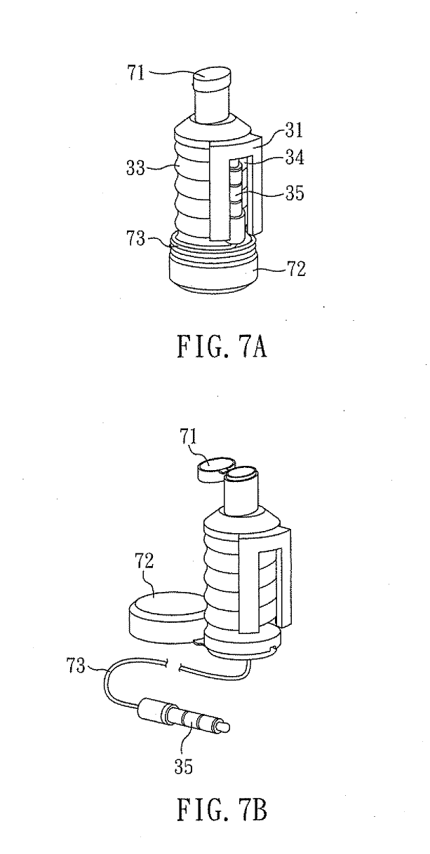

Please refer to FIG. 7A, which is a schematic diagram displaying the portable asthma detection device in the receiving status, according to the third embodiment of the present invention. In the present embodiment, the wire 73 is wound around the housing 31, wherein the housing 31 can further comprises a notch 34 for receiving the signal delivery unit 35 therein. Thus, the facility in storing the portable asthma detection device of the present invention is increased.

Please refer to FIG. 7B, which is a schematic diagram displaying the portable asthma detection device in the yet-receiving status, according to the third embodiment of the present invention. When the portable asthma detection device of the present invention is going to be used in the asthma detection, the protecting cover 71 and the wire protecting cover 72 can be opened. Then, the wound wire 73 is extended for connecting the signal delivery unit 35 and the delivery connecting unit of the smart phone. Thus, the signal delivery unit 35 is connected with the smart phone for delivering the electronic signal to the smart phone, in order to execute further processing process and computing process.

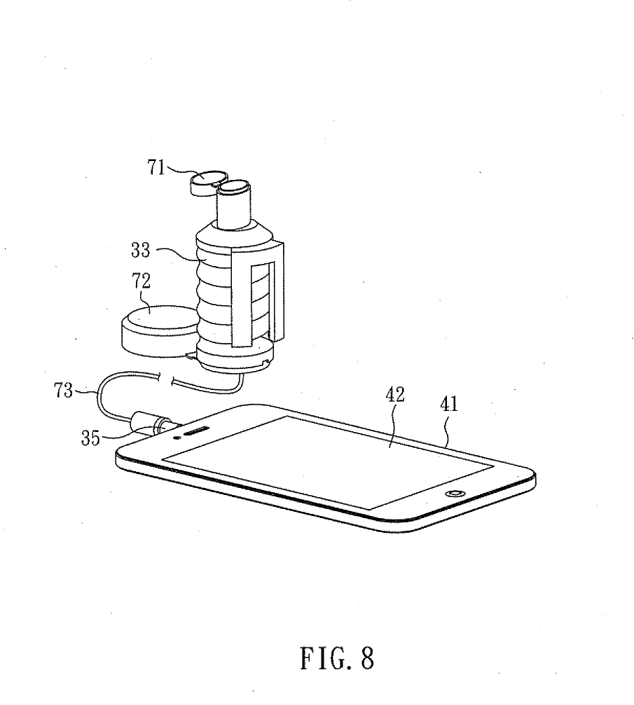

Please refer to FIG. 8, which is a schematic diagram displaying the portable asthma detection device, in cooperation with a smart phone, according to the third embodiment of the present invention. In the present embodiment, the smart phone can execute the process of computing, the process of data comparison, and the process of outputting the detection result. Besides, as the signal delivery unit 35 being connected with the smart phone 41 with the wire 73 having a certain length, the patient under test can observe the detection result displayed on the display unit 42 when he/her blows air toward the gas introducing tube. In this way, the patient under test can observe the detection result of his/her breathing dynamic, such as the detection result including the amount of the airflow, the breathing times, or the frequency of the breathing.

### Embodiment 4

In the fourth embodiment, i.e., the embodiment 4, the portable asthma detection device of the present invention, in cooperation with a drug delivery module, is provided. In this embodiment, the gas introducing tube 33 and the signal delivery unit 35 are integrated with the housing 31. The drug delivery module includes a drug container 91 and a motor (not shown in the figure). Wherein, a drug for curing or relieving the asthma disease is stored in the drug container 91. The motor is used to drive the reverse-rotation of the turbine, in order to deliver the drug to the mouth of a patient, through the gas introducing tube 33. Besides, an electronic nose or a temperature sensor can be installed at the end of the gas introducing tube 33 for detecting the chemical element, the containing element, and the temperature of the gas.

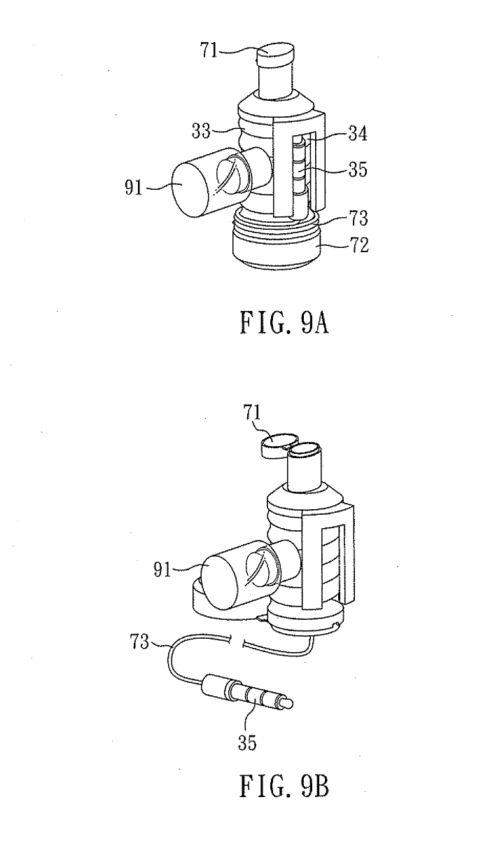

Please refer to FIG. 9A, which is a schematic diagram displaying the portable asthma detection device in the receiving status, according to the fourth embodiment of the present invention. In the present embodiment, the drug delivery module is embedded into the gas introducing tube 33, while the electronic nose is installed at the end of the gas introducing tube 33. Besides, the housing 31 further comprises a notch 34, while the signal delivery unit 35 is a foldable element. When the portable asthma detection device of the present invention is not yet used in the asthma detection, the wire is wound around the housing 31, while the signal delivery unit 35 is received in the notch 34.

Please refer to FIG. 9B, which is a schematic diagram displaying the portable asthma detection device in the yet-receiving status, according to the fourth embodiment of the present invention. When the portable asthma detection device of the present invention is going to be used in the asthma detection, the signal delivery unit 35 is extended outwardly, making the signal delivery unit 35 to be protruded from the notch 34 of the housing 31. Thus, the signal delivery unit 35 is connected with the smart phone mutually.

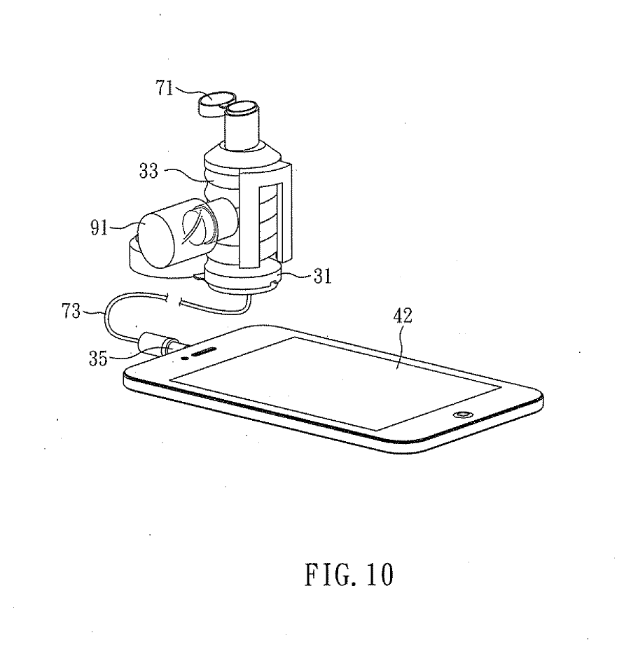

Please refer to FIG. 10, which is a schematic diagram displaying the portable asthma detection device, in cooperation with a smart phone, according to the fourth embodiment of the present invention. In the present embodiment, when the signal delivery unit is connected with the smart phone 41, the smart phone 41 can execute the process of computing, the process of data comparison, and the process of outputting the detection result, and then display the detection result, including the breathing diagram, the amount of the airflow, the breathing times, or the frequency of the breathing, on the screen thereof. In addition, as described in the previous embodiment 2, the chemical element, and the containing element of the gas detected by the electronic nose, is also displayed on the screen 42.

In the present embodiment, the smart phone can determine the current breathing condition of the patient under test, basing on the detection result. When the smart phone determines that an asthma attack is happening, the smart phone can control the motor to drive the turbine to rotate reversely, through the signal connecting unit and the signal delivery unit. Thus, the drug for curing the asthma disease, stored in the drug container, can be delivered to the mouth of the patient under test, through the gas introducing tube. In this way, the asthma attack is relieved in time. Moreover, the amount of drug to be delivered to the patient can be controlled either manually, or by the smart phone, basing on the current breathing condition just obtained through the detection.

### Embodiment 5

In the fifth embodiment, i.e., the embodiment 5, the portable asthma detection device of the present invention, in cooperation with a gas detector, is provided. In this embodiment, the portable asthma detection device has the same structure as the portable asthma detection device in the previous embodiment, i.e. embodiment 4. However, the portable asthma detection device in the present embodiment further comprises a gas detector embedded into the housing or the end of the gas introducing tube. The gas detector can include a thermoelectric cooler (T-E cooler) and at least one liquid analyzing chip.

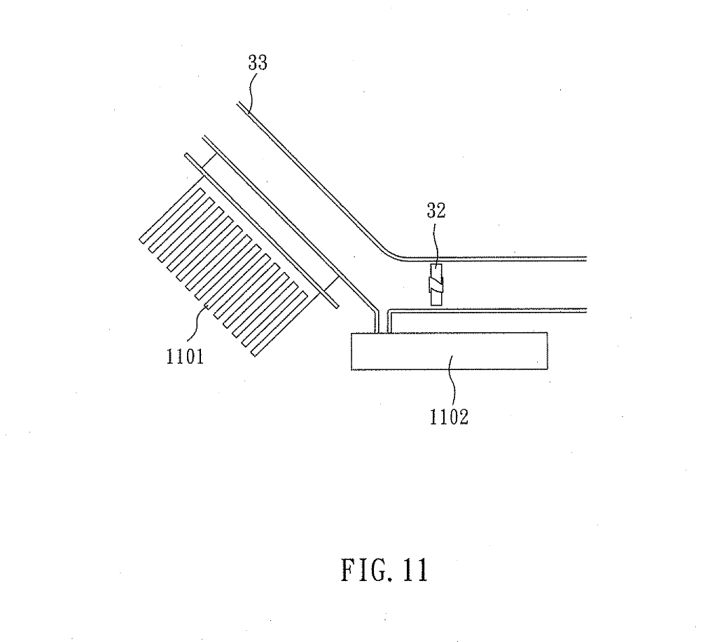

As shown in FIG. 11, a thermoelectric cooler 1101 is installed at the side of the gas introducing tube 33. When the gas is introduced to the mini airflow sensor through the gas introducing tube 33, the gas is cooled by the thermoelectric cooler 1101. Then, the liquid condensed from the gas is stayed on the surface of the thermoelectric cooler 1101. Then, the liquid is analyzed by the liquid analyzing chip, when the liquid flows through the liquid analyzing chip. The liquid analyzing chip analyzes the liquid through the electronic way, the optical way, or the chemical way. The electronic signal corresponding to the analyzing result is then transmitted to the smart phone, through the signal delivery unit and the delivery connecting unit. Thus, the smart phone can obtain the analyzing result of the liquid condensed from the gas.

### Embodiment 6

In the sixth embodiment, i.e., the embodiment 6, a stand-alone portable asthma detection device is provided. The stand-alone portable asthma detection device comprises: a housing 1201, a gas detection module, a gas introducing tube 1202, at least one input unit 1203, at least one display unit 1204, at least one delivery unit 1205. Wherein, the gas detection module includes a turbine, and a mini airflow sensor. Multiple input units 1203 can be installed on the housing for inputting the control instructions to the stand-alone portable asthma detection device of the present invention. Besides, the delivery unit 1205 can be an antenna for the wireless network transmission function and the positioning function.

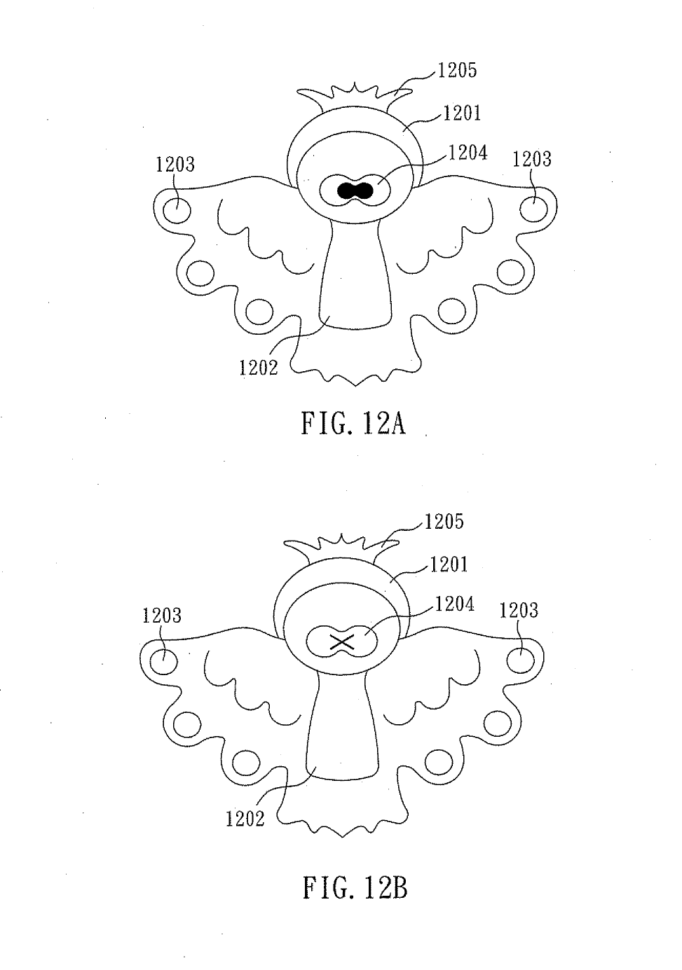

Please refer to FIG. 12A, and FIG. 12B, which are the schematic diagrams displaying the stand-alone portable asthma detection device of the present invention. The operation of the stand-alone portable asthma detection device, such as the turn-on, the detection, the output of detection result, is controlled through the input unit 1203 in the button form. The patient under test can blow air toward the gas introducing tube 1202, enabling the stand-alone portable asthma detection device to execute the asthma detection. When the computing module of the stand-alone portable asthma detection device determines that the breathing condition of the patient under test is below the normal range, a simplified detection result is output on the display unit 1204, such as an electronic paper or a display screen, in the form “X”, as shown in FIG. 12B. On the other hand, when the computing module of the stand-alone portable asthma detection device determines that the breathing condition is within the normal range, another simplified detection result is output on the display unit 1204, in the normal form shown in FIG. 12A.

The stand-alone portable asthma detection device of the present invention can transmit the detection result, along with the geographical information of the detection, to the database, the hospital, or the medical personnel, through the wireless transmission function and the positioning function provided by the smart phone, for obtaining a complete, comprehensive, and efficient monitoring and managing on the asthma disease.

Moreover, as the portable asthma detection device of the present invention in the previous embodiments, from embodiment 2 to embodiment 5, the stand-alone portable asthma detection device of the present invention can be modified, or be in cooperation with a drug delivery module or a gas detector, for providing more function and flexibility in the asthma detection.

Although the present invention has been explained in relation to its preferred embodiment, it is to be understood that many other possible modifications and variations can be made without departing from the spirit and scope of the invention as hereinafter claimed.

What is claimed is:

1. A portable asthma detection device, comprising:
a housing;
a gas detection module, which comprising:
a turbine; and
a mini airflow sensor, for detecting the rotation of the turbine;
a gas introducing tube, connecting with the housing and introducing the gas into the gas detection module; and
a signal delivery unit, connecting with the housing mutually.

2. The portable asthma detection device as claimed in claim 1, further comprising a microprocessor, wherein the microprocessor comprises:
a delivery connecting unit, for connecting the signal delivery unit with the microprocessor, enabling the delivery of the electronic signal output from the gas detection unit, to the microprocessor;
a computing module, for transforming the electronic signal into a detection result; and
at least one display unit, for displaying the detection result.

3. The portable asthma detection device as claimed in claim 1, wherein the gas detection module is an electro-magnetic type sensing module, an optical coupling type sensing module, or a power-generating type sensing module.

4. The portable asthma detection device as claimed in claim 1, further comprising at least one selected from the group consisting of an electronic nose, a temperature sensor, and a gas detector.

5. The portable asthma detection device as claimed in claim 4, wherein the electronic nose is connected with the gas introducing tube for detecting the chemical element and the gas molecule in the gas.

6. The portable asthma detection device as claimed in claim 4, wherein the temperature sensor is connected with the gas introducing tube for detecting the temperature of the gas.

7. The portable asthma detection device as claimed in claim 4, wherein the gas detector includes a thermoelectric cooler or at least one liquid analyzing chip.

8. The portable asthma detection device as claimed in claim 4, wherein the electronic nose is used to detect the nitric oxide, the carbon monoxide, or the carbon dioxide in the gas.

9. The portable asthma detection device as claimed in claim 1, wherein the gas introducing tube, the gas detection module, and/or the signal delivery unit are integrated with the housing.

10. The portable asthma detection device as claimed in claim 1, further comprising a drug delivery module, wherein the drug delivery module includes:
a drug container, for storing a drug for curing asthma disease; and
a motor, for driving the rotation of the turbine, in order to deliver the drug to the mouth of a patient, through the gas introducing tube.

11. The portable asthma detection device as claimed in claim 1, wherein a notch is further installed at the exterior side of the housing for receiving the signal delivery unit therein.

12. The portable asthma detection device as claimed in claim 1, further comprising a wire being installed between the signal delivery unit and the housing.

13. The portable asthma detection device as claimed in claim 2, wherein the microprocessor is a smart phone.

14. The portable asthma detection device as claimed in claim 2, wherein the microprocessor further comprises at least one system selected from the group consisting of a position system, a wireless transmission system, and a remote monitoring system.

15. A stand-alone portable asthma detection device, comprising:
a housing;
a gas detection module, which comprising:
a turbine; and
a mini airflow sensor, for detecting the rotation of the turbine;
a gas introducing tube, connecting with the housing and introducing the gas into the gas detection module;
at least one input unit, being installed on the housing;
at least one display unit, being installed on the housing and used for displaying a detection result; and
at least one delivery unit, for providing a positioning function and a wireless network function.

16. The stand-alone portable asthma detection device as claimed in claim 15, wherein the gas detection module is an electro-magnetic type sensing module, an optical coupling type sensing module, or a power-generating type sensing module.

17. The stand-alone portable asthma detection device as claimed in claim 15, further comprising at least one selected from the group consisting of an electronic nose, a temperature sensor, and a gas detector.

18. The stand-alone portable asthma detection device as claimed in claim 17, wherein the electronic nose is connected with the gas introducing tube for detecting the chemical element and the gas molecule in the gas.

19. The stand-alone portable asthma detection device as claimed in claim 17, wherein the temperature sensor is connected with the gas introducing tube for detecting the temperature of the gas.

20. The stand-alone portable asthma detection device as claimed in claim 17, wherein the gas detector includes a thermoelectric cooler or at least one liquid analyzing chip.

21. The stand-alone portable asthma detection device as claimed in claim 17, wherein the electronic nose is used to detect the nitric oxide, the carbon monoxide, or the carbon dioxide in the gas.

22. The stand-alone portable asthma detection device as claimed in claim 15, wherein the gas introducing tube, the gas detection module, and/or the signal delivery unit are integrated with the housing.

23. The stand-alone portable asthma detection device as claimed in claim 15, further comprising a drug delivery module, wherein the drug delivery module includes:
a drug container, for storing a drug for curing the asthma disease; and
a motor, for driving the rotation of the turbine, in order to deliver the drug to the mouth of a patient, through the gas introducing tube.
